(19) United States
(12) Patent Application Publication
(10) Pub. No.: US 2020/0038824 A1
(43) Pub. Date: Feb. 6, 2020

(54) SMART MIXING SYSTEM FOR PERSONALIZED COSMETICS, AND ASSOCIATED SYSTEMS AND METHODS

(71) Applicant: L'Oreal, Paris (FR)

(72) Inventors: Grégoire Charraud, Saint-Ouen (FR); Helga Malaprade, Saint-Ouen (FR); Haruna Peyret, San Francisco, CA (US); Guive Balooch, New York, NY (US)

(73) Assignee: L'Oreal, Paris (FR)

(21) Appl. No.: 16/051,142

(22) Filed: Jul. 31, 2018

(51) Int. Cl. B01F 13/10; A61B 5/00; B01F 15/00

(52) U.S. Cl. CPC B01F 13/1063; A61B 5/442; A45D 2044/007; B01F 2215/0031; B01F 15/00207

(57) ABSTRACT

In some embodiments, the present disclosure provides a personalized cosmetic formula preparing system, a method of operating the system, and computer readable instructions for implementing the method. The system determines a personalized cosmetic formula for a consumer. In an embodiment, the determination is based on the consumer's preferences, lifestyle, and cosmetic attributes. The system comprises one or more beauty assessment devices, a digital hub in communication with the one or more beauty assessment devices, a dispenser, and a mixer. The system may be used directly in communication with the consumer, or may automatically obtain the formula from other sources. The companion app may control the constituent elements of the system remotely via the digital hub. The formula preparing system may cause a personalized cosmetic formula to be prepared and packaged without complex or expensive equipment and without extensive cosmetic technical training for the operator of the system.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

The inventive technology is directed to personalized cosmetics products prepared at the point-of-sale, in a short time, and with minimal complexity. The inventive technology may be implemented as a system, a method of operation, and/or a software application. Conventional approaches to production and sales of cosmetic products focus on either fully-customized cosmetic formulations or fully-prepared products sold over-the-counter. No streamlined customization experience is currently available for delivering a personalized cosmetic product in a retail setting. The inventive technology provides a personalized cosmetic product in a retail setting. The personalized product may be prepared in a fraction of the time normally required for a custom product and at a fraction of the typical price. Furthermore, the inventive technology may incorporate model predictive aspects that anticipate the consumer's cosmetic needs in advance.

In some embodiments, the inventive technology includes an assessment of cosmetic formula parameters, and a personalized cosmetic product is prepared according to a personalized cosmetic formula. In some embodiments, inventive method proceeds from a beauty assessment of cosmetic formula parameters, through the development of the personalized cosmetic formula, and to the dispensing of one or more active ingredients into a formula container. In some embodiments, the formula container may be mixed before being provided to the consumer.

The mixer may implement an optimized mixing profile that is paired to one or more physical properties of the ingredients that were dispensed into the personalized cosmetic formula container. For example, in one aspect, the viscosity of the contents of the formula container may affect the mixing profile implemented by the mixer. In some embodiments, information, including the results of the beauty assessment and the personalized cosmetic formula, may be saved on a cloud storage system.

In some embodiments, a system for preparing and packaging a personalized cosmetic formula includes one or more beauty assessment devices. In one aspect, the one or more beauty assessment devices individually have one or more sensors that measure, detect, and/or receive one or more beauty parameters. In one aspect, the one or more beauty assessment devices measure the beauty parameters, and provide the measurements as personalized cosmetic formula information to the other constituent elements of the system. In one aspect, the system includes a cloud storage system, performing at least one of: receiving personalized cosmetic formula information, storing the personalized cosmetic formula information, and communicating the information to an external recipient. In one aspect, information stored in the cloud storage system is processed by a formulation engine to reduce a number of degrees of freedom measured by the one or more beauty assessment devices and to determine a routine of personalized cosmetic formulas. In one aspect, the routine is a series of personalized cosmetic formulas for implementing an incremental treatment over time by, for example, using a graduated dose of an active ingredient.

In some embodiments, the system includes a digital hub that receives data from the one or more beauty assessment devices and sends dispensing instructions to the dispenser and mixing instructions to the mixer. In one aspect, the system includes a store screen. In one aspect, the store screen receives personalized cosmetic formula information from the digital hub and displays the personalized cosmetic formula information visually. In one aspect, an anticipated cosmetic result of the routine is superimposed on a contemporaneous image of a consumer using the store screen. The anticipated cosmetic result may display a predicted therapeutic effect of a medicated personalized cosmetic formula, or a predicted cosmetic effect. In one aspect, the contemporaneous image of a consumer is a digital photograph showing at least one of the skin, face, and hair of the consumer.

In some embodiments, the system includes a dispenser having a plurality of mono-dose containers and a cosmetic formula packaging container. The individual mono-dose containers hold at least one ingredient. In one aspect, the cosmetic formula packaging container is pre-filled with at least one ingredient.

In one aspect, the system includes a compact printer engraving machine that receives label information from the digital hub and creates a label for the personalized cosmetic formula packaging container.

In some embodiments, the system includes a mixer that is compatible with the cosmetic formula packaging container shape. In one aspect, the mixer includes an interlock. In one aspect, the mixer includes a weighing stage. In one aspect, the weighing stage records a measured weight of the cosmetic formula packaging container after dispensing and before mixing to compare the measured weight against a calculated formula weight for the cosmetic formula packaging container, and to activate the interlock to interlock the mixer when the measured weight and the calculated formula weight are substantially unequal. In some embodiments, the measured weight and the calculated formula weight are substantially unequal when the difference between the two exceeds a threshold value, expressible as a percentage of the calculated formula weight. In one aspect, the threshold value is in the range of approximately 0.1%-10%. In one aspect, the mixer includes one or more sensors 58 in communication with the digital hub. In one aspect, the mixer implements a mixing cycle paired to the personalized cosmetic formula that is being prepared.

In some embodiments, the method includes conducting the beauty assessment. In one aspect, the beauty assessment determines values of cosmetic parameters describing aesthetic, therapeutic, and lifestyle aspects that are addressable by the personalized cosmetic formula.

In some embodiments, the method includes communicating one or more results of the beauty assessment to the digital hub. In one aspect, the digital hub implements a companion app, which is a non-transitory computer readable medium having computer executable instructions stored thereon that, in response to execution by one or more processors of a computing device, cause the computing device to implement the method as described herein.

In some embodiments, the method includes creating a personalized cosmetic formula. In one aspect, creating the personalized cosmetic formula includes processing the results of multiple beauty assessments in the formulation engine to produce the routine. In one aspect, the method includes attributing a unique identifier code to the customized formula. In some embodiments, the method includes communicating the personalized cosmetic formula to a dispenser, a mixer, and a compact printer engraving machine.

In some embodiments, the method includes: preparing the personalized cosmetic product by dispensing one or more mono-doses of at least one active ingredient into the cosmetic formula packaging container according to the personalized cosmetic formula, mixing the personalized cosmetic product, and labelling the personalized cosmetic product packaging container.

In some embodiments, the method includes recording a measured weight of the cosmetic formula packaging container before mixing, comparing the measured weight against a calculated formula weight for the cosmetic formula packaging container, and interlocking the mixer when the measured weight and the calculated formula weight are substantially unequal.

### DESCRIPTION OF THE DRAWINGS

The foregoing aspects and attendant advantages of the inventive technology will become more readily appreciated as the same become better understood by reference to the following detailed description, when taken in conjunction with the accompanying drawings, wherein:

FIG. 1 is a schematic diagram of a system in accordance with the present disclosure;

FIG. 2 is a block diagram of a system in accordance with the present disclosure; and

FIG. 3 is a flowchart of a method of creating and preparing a personalized cosmetic formula according to the present disclosure.

### DETAILED DESCRIPTION

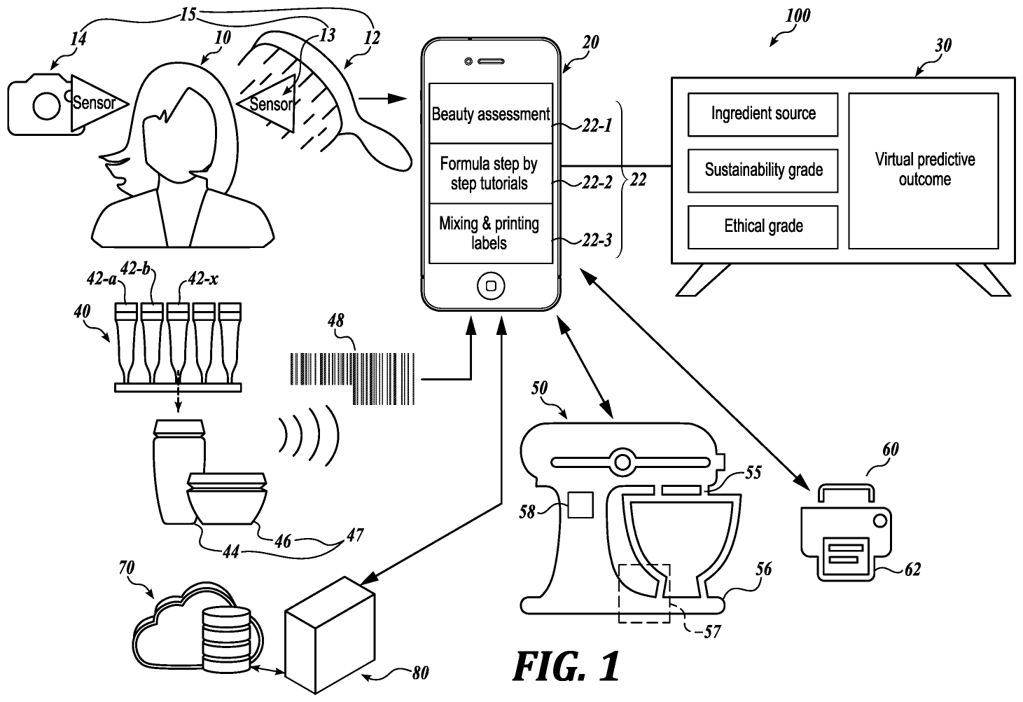

The following disclosure describes various embodiments of systems and associated methods for preparing personalized cosmetic formulas. A person skilled in the art will also understand that the technology may have additional embodiments, and that the technology may be practiced without several of the details of the embodiments described below with reference to FIGS. 1-3.

At present, personalized cosmetic products are financially out of reach for many consumers. Custom formulations are prepared by skilled cosmetic experts, specially trained to follow precise procedures and to operate complex blending machines. The cosmetics produced in this manner require considerable time to prepare by a cosmetics expert and are typically much more expensive than ready-made cosmetics purchased over the counter. Nonetheless, there is considerable demand for personalized cosmetics at an accessible price. For example, many consumers are sensitive to fragrances that are common ingredients in prepared cosmetics. Such consumers are faced with a limited selection of cosmetics that omit scented ingredients, which may not provide the same cosmetic or therapeutic effects, or they must pay for custom formulations. In another example, custom formulations are not available at every retail location, unlike prepared cosmetics. Therefore, a consumer of custom formulations must travel with a sufficient supply to last until his or her return, or must purchase prepared cosmetics in the interim period. Systems and methods for creating personalized cosmetic products are described herein, employing equipment and cosmetic technical training streamlined for use at the point-of-sale.

FIG. 1 is a schematic diagram of a system 100 in accordance with the present disclosure. The system 100 may determine and prepare a personalized cosmetic formula. In an embodiment, the system 100 includes: one or more beauty assessment devices 15, a digital hub 20, a store screen 30, a dispenser 40, a mixer 50, a compact printer engraving machine 60, a cloud storage system 70, and a formulation engine 80. In some embodiments, one or more beauty assessment devices 15 are used in a step-by-step tutorial 22-2 with a consumer 10. Non limiting examples of such beauty assessment devices 15 include a hair brush 12, an electronic brush sensor 13, a skin sensor 14, and the like. The beauty assessment devices 15 can be used to collect information about personal cosmetic needs of the consumer 10. Non limiting examples of such information include skin condition, age, skin color, lifestyle, typical UV exposure, personal preferences, and the like. In an embodiment, the system 100 further includes a digital hub 20 having a non-transitory computer readable medium with instructions and data stored thereon that, in response to execution by one or more processors of a computing device, cause the computing device to implement the method as described herein (hereinafter referred to as a companion app 22). The companion app 22 provides the instructions and data for preparing the personalized cosmetic formula and communicates the personalized cosmetic formula to other elements of the system 100. Non-limiting examples of such instructions and data include: a report on the beauty assessment 22-1, step-by-step tutorials 22-2, a preview of mixing and printing labels 22-3, and the like. In operation, a cosmetic technician may receive and input data and instructions using the companion app 22.

In some embodiments, the digital hub 20 communicates with a store screen 30, which presents information produced by the elements of the system 100. Non-limiting examples of such information include source information about the ingredients used in the personalized cosmetic formula, a sustainability grade for the personalized cosmetic formula, an ethical grade for the personalized cosmetic formula, a virtual predictive outcome superimposing a predicted result of cosmetic treatment onto a contemporaneous image of the consumer 10, and the like.

A dispenser 40 combines active ingredient mono-doses 42-x with inactive ingredients (not shown), according to the personalized cosmetic formula. In an embodiment, the digital hub 20 provides the personalized cosmetic formula to the dispenser 40. In some embodiments, the dispenser 40 includes a plurality of active ingredient mono-dose 42-x containers and a formula bottle 44, a formula jar 46, or a container of another form factor (hereinafter collectively referred to as a formula container 47). Non-limiting examples of the active ingredient mono-doses 42-x include fragrance mono-doses 42-a, pigment mono-doses 42-b, medicament mono-doses, essential oil mono-doses, plant extract mono-doses, and the like. In some embodiments, active ingredient mono-doses 42-x further include a diluent in addition to the active ingredient. For example, a fragrance mono-dose 42-a may contain an essential fragrance diluted in an odor-neutral oil. In some embodiments, the formula is attributed a formula identification code 48 that the dispenser 40 communicates to the digital hub 20, whereupon the consumer 10 may receive the formula identification code 48. In some embodiments, the formula identification code 48 includes information about the ingredients and their proportions in the personalized cosmetic formula. In some embodiments, the formula identification code 48 also includes information about the consumer 10.

In some embodiments, the mixer 50, which is in communication with the digital hub 20, receives the formula container 47. In some embodiments, the mixer 50 may house the formula container and mix its contents after the formula container 47 has been closed, without the mixer directly contacting the contents. For example, the mixer 50 may shake the formula container 47 to mix its contents.

In some embodiments, the mixer 50 further includes a weighing stage 56, which can be used to determine a measured weight of the formula container 47 after the dispenser 40 has dispensed ingredients according to the personalized cosmetic formula. The calculated formula weight of the formula container 47 can be determined by summing the weight of the formula container 47 when empty with the individual weights of the ingredients of the personalized cosmetic formula, identified by the formula identification code 48. Because slight variation in the measured weight may occur, in some embodiments the mixer 50 includes an interlock 57 to prevent mixing if the measured weight is substantially unequal from the predicted weight. In some embodiments, the measured weight and the calculated formula weight are substantially unequal when the difference between the two exceeds a threshold value, expressible as a percentage of the calculated formula weight. In one aspect, the threshold value is in the range of approximately 0.1%-10%. In some embodiments, the threshold value is in the range of 0.1%-2%. In some embodiments, the threshold value is in the range of 0.2%-5%. In some embodiments, the threshold value is in the range of 5%-10%. In some embodiments, the threshold value is equal a unit weight of the contents of an individual active ingredient mono-dose 42-x.

In some embodiments, the formula preparing system 100 includes a compact printer engraving machine 60, in communication with the digital hub 20. The compact printer engraving machine 60 may include at least one of a printer or an engraver, and may prepare packaging label information 62 for cosmetic formula packaging, such as the formula container 47 or a cardboard enclosure (not shown).

In some embodiments, the formula preparation system 100 includes a cloud storage system 70 in communication with the digital hub 20. In some embodiments, the cloud storage system 70 stores information including the formula identification code 48, consumer purchase history, and consumer product preferences. In some embodiments, the cloud storage system 70 links this information to a consumer account and/or profile.

As understood by one of ordinary skill in the art, a “cloud storage system” as described herein may be any suitable device configured to store data for access by a computing device. An example of the cloud storage system 70 is a relational database management system (DBMS) executing on one or more computing devices and being accessible over a high-speed network. However, other suitable storage techniques and/or devices capable of providing the stored data in response to queries may be used, and the computing device may be accessible locally instead of over a network, or may be provided as a cloud-based service. The cloud storage system 70 may also include data stored in an organized manner on a computer-readable storage medium.

In some embodiments, the formula preparation system 100 includes a formulation engine 80 with machine-learning software for recognizing and promoting factors responsible for desired outcomes. Non-limiting examples of such factors are: increased consumer 10 satisfaction, increased sales, increased treatment effectiveness, decreased negative outcomes, and the like. In some embodiments, the formulation engine 80 reduces a number of degrees of freedom in the beauty assessment, after recognizing the influential factors responsible for the desired outcomes. The formulation engine 80 may reduce the number of degrees of freedom by eliminating, or reducing the statistical weight of, one or more parameters of the beauty assessment 22-1 or by removing one or more steps from the step by step tutorial 22-2. In some embodiments, the formulation engine 80 recognizes that a consumer 10 is, for example, not interested in hair products, and removes the data produced by the brush sensor 12 from the beauty assessment. In some embodiments, the engine receives information from the companion app 22 or the cloud storage system 70 including: a location of the point-of-sale, a timeframe of anticipated use, the results of the beauty assessment, the beauty assessment parameters, the formula identification code 48, the personalized cosmetic formula being prepared, consumer interaction data, and the like.

Consumer interaction data may include: aesthetic preferences, such as trends, treatment information, self-reporting of reactions to various active ingredients, or other categories including unqualified “yes” or “no” preferences, unsolicited expressions of satisfaction or dissatisfaction, statements of preference for competing cosmetic products, and the like. In some embodiments the formulation engine 80 is designed to develop generalized models for parameters that display a sufficiently low variance over time or across a designated set of personalized cosmetic formulas. For example, the formulation engine 80 may determine a season or time of year to begin including UV-absorbing active ingredients in higher concentrations. The formulation engine 80 also may determine when a particular color or a set of colors has gained broad popularity within a given timeframe and whether to include the same or similar colored pigments in the personalized formula being prepared.

In some embodiments, the formulation engine 80 prepares a routine including more than one personalized cosmetic formula to be provided as a series of personalized cosmetic formula products, to achieve a desired cosmetic result over a period of time. In some embodiments, the routine is designed to incrementally increase the concentration of an active ingredient, such as a medicament, between a first personalized cosmetic formula in the routine and a second personalized cosmetic formula in the routine, to achieve a desired therapeutic effect over the course of the routine. One of ordinary skill in the art will recognize that the implementations described herein are illustrative examples, and other examples are readily implemented without departing from the scope of the present disclosure.

In general, the word “engine,” as used herein, refers to software and algorithms embodied in hardware or software instructions, which can be written in a programming language, such as C, C++, COBOL, JAVA™, PHP, Perl, HTML, CSS, JavaScript, VBScript, ASPX, Microsoft .NET™, PYTHON, and/or the like. An engine may be compiled into executable programs or written in interpreted programming languages. Software engines may be callable from other engines or from themselves. Generally, the engine described herein refers to logical modules that can be merged with other engines, or can be divided into sub-engines. The engines can be stored in computer-readable medium or computer storage device and be stored on and executed by one or more general purpose computers, thus creating a special purpose computer configured to provide the engine or the functionality thereof.

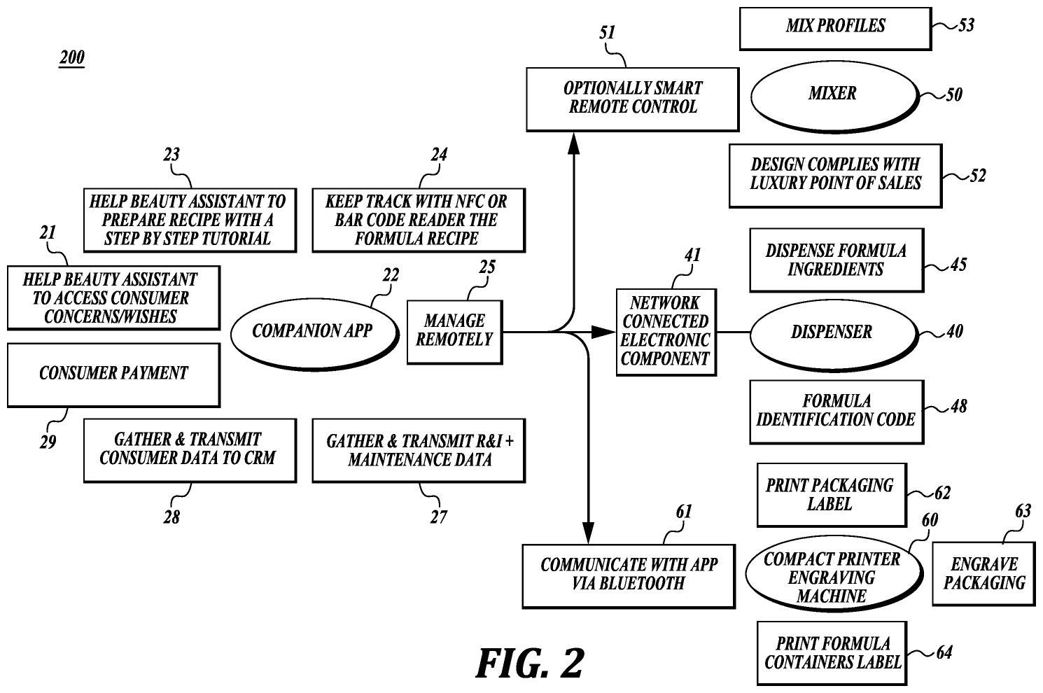

FIG. 2 is a block diagram of a formula preparing system 200 in accordance with the present disclosure. The formula preparing system 200 may implement beauty assessment tutorials, remotely manage elements of the formula preparing system 200, and track personalized cosmetic formula preparation. Though some aspects are illustrated and described as relating to personalized cosmetic formulas, in some embodiments, products other than personalized cosmetic formulas may be processed by the system 200.

As shown, the formula preparing system 200 includes the companion app 22, the dispenser 40, the mixer 50, and the compact printer engraving machine 60. These components may communicate to each other via a network (not shown), which may include suitable communication technology including, but not limited to, wired technologies such as DSL, Ethernet, fiber optic, USB, and Firewire; wireless technologies such as WiFi, WiMAX, 3G, 4G, LTE, and Bluetooth; and the Internet.

In some embodiments, the companion app 22 helps a cosmetic technician to assess the consumer's wishes and concerns, as shown in box 21. Such wishes and concerns may include cosmetic preferences, trend and lifestyle choices, therapeutic sensitivities, allergies, and the like. In some embodiments, the companion app 22 presents a questionnaire, displayed on the digital hub 20, to determine consumer 10 wishes and concerns. In some embodiments, the cosmetic technician records responses given by the consumer 10 to questions presented by the companion app 22, and posed to the consumer 10 orally, in writing, or both.

In some embodiments, the companion app 22 interacts with other components of the formula preparing system 200. Typically, the companion app 22 runs on a mobile computing device such as a smartphone or a tablet computing device. However, any other suitable type of computing device capable of communicating via the network and presenting a user interface, including but not limited to a desktop computing device, a laptop computing device, and a smart watch, may be used.

In some embodiments the companion app 22 helps the cosmetic technician to prepare a recipe with the step-by-step tutorial 22-1, as shown in box 23. The tutorial includes directions on using the beauty assessment devices 15 such as the hair brush 12 or the skin sensor 14 to conduct the beauty assessment 22-1, directions on dispensing ingredients, operating the mixer 50, and the like.

In some embodiments, the companion app 22 tracks the progress of the personalized cosmetic formula through its formulation, as shown in box 24. In some embodiments, the formula identification code 48 is used to track progress, with the other units of the formula preparing system 200 in communication with the companion app 22 via the digital hub 20. In some embodiments, a barcode on the formula container 47 is assigned to the personalized cosmetic formula as the formula identification code 48. In some embodiments, the companion app 22 is manually updated with progress information, and the formula identification code 48 is assigned manually.

In some embodiments, the companion app 22, in communication through the digital hub 20 with the dispenser 40, the mixer 50, and the compact printer engraving machine 60, remotely manages their operation, as shown in box 25. In some embodiments, managing operation includes sending commands via the digital hub 20 to a network connected electronic component 41, via an operationally smart remote control 51, or via Bluetooth 61. In some embodiments, the companion app 22 sends commands to activate and deactivate the dispenser 40, mixer 50 and/or the compact printer engraving machine 60. In some embodiments, managing operation includes remote control of the units throughout the process including, but not limited to, automatically or via electronic command: selecting a formula container 47; controlling dispensing 45 of the mono-dose containers 42-x into the formula container 47; selecting a mixing profile 53; performing a weight check; activating the mixer 50; sending label information to the compact printer engraving machine 60; activating the compact printer engraving machine 60; and deactivating the elements of the formula preparing system 200.

In some embodiments, the companion app 22, in communication with the formula preparing system 200 via the digital hub 20, receives data from the formula preparing system 200 and transmits data to the cloud storage system 70, as shown in box 27 and box 28. In some embodiments, the data includes research and innovation (R&I) and maintenance data, wherein the companion app 22 receives alerts and reports from the constituent elements in the formula preparing system 200 including operation errors, process start notifications, process end notifications, machine status messages, and the like. In some embodiments, the data includes consumer data, such as consumer 10 profiles, purchase history, product preferences, and the like.

In some embodiments, the companion app 22, in communication with the digital hub 20, processes payment information at the point of sale. As shown in box 29, the companion app 22 may receive payment information from the consumer 10 and may send it for further processing, including to the cloud-storage system 70

In some embodiments, the mixer 50 includes computer readable instructions stored in nonvolatile memory that, when executed, enable remote operation by the companion app 22. Remote operation occurs in communication with the companion app 22 via the digital hub 20, as shown in box 51. In some embodiments, the aesthetic design of the mixer 50 complies with luxury point of sales, as shown in box 52. Aesthetic design of the mixer 50 includes the visual profile of the physical enclosure of the mixer 50, acoustic characteristics of the mixer 50 when in operation, materials selected for the construction of the mixer 50, and the like. In some embodiments, the mixer implements mix profiles, as shown in box 53, described in more detail below.

In some embodiments, the compact printer engraving machine 60 includes computer readable instructions stored in nonvolatile memory that, when executed, enable remote operation. Remote operation occurs in communication with the companion app 22 via the digital hub 20, as shown in box 61. In some embodiments, as previously described, the compact printer engraving machine 60 is capable of engraving packaging directly, as shown in box 63, and of printing labels for bottles and packaging, as shown in boxes 62 and 64, respectively.

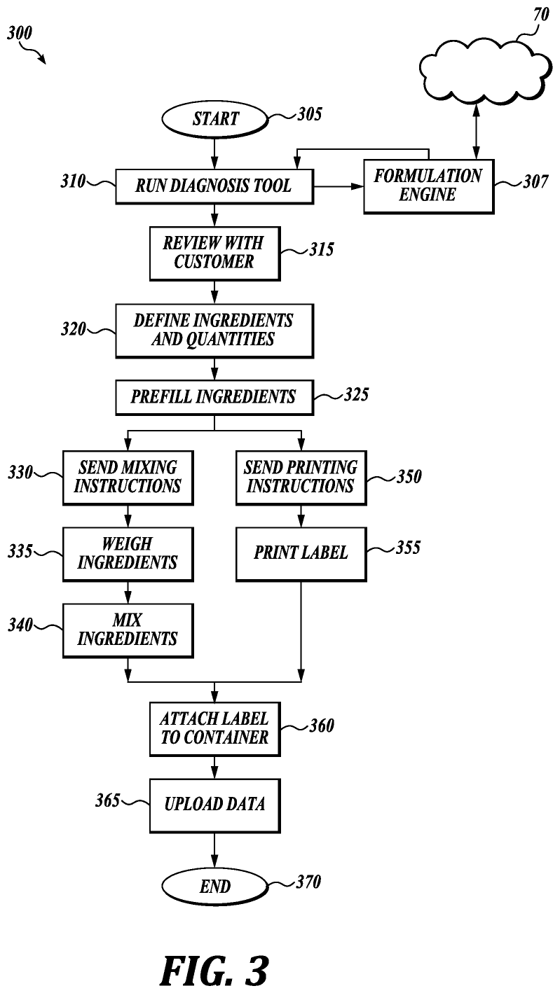

FIG. 3 is a flowchart that illustrates an example embodiment of a method 300 of preparing a personalized cosmetic formula according to various aspects of the present disclosure. The method starts in block 305 and proceeds to block 310, where the companion app 22 runs a diagnosis tool implementing the beauty assessment of the consumer 10. The diagnosis tool may assess product preferences including, but not limited to, product form factor, lifestyle fit, and persistence. The diagnosis tool may assess cosmetic preferences including: the consumer 10 skin and hair needs, aesthetic preferences, such as fragrance, pigment, environmental and ethical stance, and tolerance for various active ingredients, and the like. For example, the consumer 10 may specify a preference for a first shade in a persistent formula for daytime use, and a preference for a second shade in a transitory formula for evening use.

At block 315, the consumer or the cosmetic technician reviews the beauty assessment 22-1 and may provide further information including, but not limited to, final approval of the assessment, and acceptance of an anticipated price of the finished product. Following review with the consumer 10, the method 300 proceeds to block 320, including defining of the personalized cosmetic formula. The personalized cosmetic formula includes information such as: a list of constituent ingredients to be combined, a list of individual quantities of the constituent ingredients, an order of addition of the ingredients, and the like. The individual quantities of the constituent ingredients may be expressed in a common unit, such as milliliters or milligrams, or in another form, such as a number of mono-doses.

At block 325, the dispenser 40 prefills a formula container 47 according to the personalized cosmetic formula defined in block 320. The formula container 47 will fit a form factor suitable for the product being provided. For example, form factors may include the formula bottle 44, the formula jar 46, a cosmetic formula tube, and the like. During block 325, the operator of the formula preparing system 100 may operate the dispenser 40 directly, or may operate the dispenser indirectly. In some embodiments, the operator may accomplish the step of prefilling ingredients entirely manually, by hand-dispensing into the formula container 47 a number of mono-doses 42-x according to block 320. In some embodiments, the companion app 22 operates the dispenser 40 remotely, as described with reference to FIG. 2. Remotely operating the dispenser 40 may include processes such as: automatically selecting and dispensing one or more mono-dose 42-x containers, automatically providing a formula container 47, manually feeding mono-dose 42-x containers into the dispenser 40 that automatically selects the appropriate formula container 47, and manually providing a formula container 47 that is automatically filled at least partially with at least one active ingredient mono-dose 42-x. One of ordinary skill in the art will recognize that the implementations described herein are illustrative examples, of which many others are implementable without departing from the scope of the present disclosure

Blocks 330 and block 350 include communicating instructions to the constituent elements of the formula preparing system 100. In the illustrated embodiment, the method 300 presents block 330 and block 350 in parallel, but the actions represented therein need not be taken at the same time. In box 330, sending mixing instructions to the mixer 50 includes, but is not limited to, manually entering a mixing profile into the mixer 50, and transmitting mixing instructions 53 via the companion app 22. Mixing instructions 53 may include a dynamic mixing cycle specifically paired to the formula being prepared, a steady mixing profile applied for a set duration, and a dynamic mixing profile, varying in at least one of power or duration, wherein the parameters are set independently of the formula being prepared. In a preferred embodiment, the mixing duration is in the range of 20 seconds-2 minutes. In some embodiments, the mixing duration is determined at least in part by requirements drawn to product uniformity and homogeneity. In some embodiments, properties, such as average viscosity and colloidal heterogeneity of the personalized cosmetic ingredients, determine, at least in part, the mixing parameters. In some embodiments, the mixing instructions include information on the shape of the formula container 47. The shape of the formula container 47 may affect, among other aspects of the mixer 50 operation, the range of motion of the formula container 47, the maximum force needed, and the mixing speed.

In box 350, sending printing instructions includes, but is not limited to, manually entering consumer 10 information, formula information, and product information into the compact printer engraving machine 60, and transmitting the same or similar information via the companion app 22. The method 300 then proceeds to box 355, whereupon the compact printer engraving machine 60 prepares a label according to the printing instructions. In some embodiments, the compact printer engraving machine 60 prepares the packaging label 62, the container label 64, and the packaging engraving 63, individually presenting information according to instructions provided in box 350. In some embodiments, the packaging label 62 includes the formula identification code 48, consumer identification information, and a visual design. The engraving may display, among other things, the name of the consumer 10, the formula identification code 48, and the date of preparation of the product. The bottle label 64 may display, among other things, the name of the consumer 10 and a name and a type of the product contained in the container.

In box 335, the mixer 50, including the weigh stage 56, determines a measured weight of the filled formula container 47. The measured weight is communicated to the digital hub 20, where it is compared to a calculated weight of the formula and the formula container 47, based on the personalized cosmetic formula. In some embodiments, the calculated weight includes the weights of the ingredients, the container, and the lid of the container. In some embodiments, the mixer 50 interrupts the method 300 from proceeding to box 340 when the measured weight is substantially unequal from the calculated weight, as previously described with respect to FIG. 1. In some embodiments, the companion app 22 determines a missing ingredient by calculating a difference between the calculated weight and the measured weight and comparing the difference to the individual weights of the contents of the individual active ingredient mono-dose 42-x containers dispensed at box 325.

In box 340, the mixer 50 may mix the ingredients using a mixing tool 55 in direct contact with the cosmetic ingredients, which may be discarded following mixing. In some embodiments, the mixing tool 55 may be a combined mixing/applicator tool incorporated into the lid or top portion of the formula container 47. In some embodiments, the mixer 50 mixes the ingredients indirectly by agitating the container once the container has been closed, sealed, or otherwise covered.

In box 360, the prepared label is attached to the container. In some embodiments, the label is attached manually. In some embodiments, the label is an engraving, made into the formula container 47 by the compact printer engraving machine 60. In some embodiments, the engraving is made before proceeding to box 325. In some embodiments, the compact printer engraving machine 60 engraves the label information 24 onto the formula container 47 before the personalized cosmetic formula ingredients are dispensed into the formula container 47. In some embodiments, the mixer 50 further includes a label applicator tool (not shown) and the mixer 50 automatically attaches the label.

In box 365, the companion app 22 that is implemented on the digital hub 20 sends data to the cloud storage system 70. In some embodiments, the digital hub 20 uploads the formula identification code 48 and other information including: consumer 10 identification information, purchase information, point-of-sale location, cosmetic formula routine information, and the like. Method 300 ends in block 370.

In some embodiments, the method 300 includes the digital hub 20 calling the formulation engine 80, in box 307, prior to running the diagnosis tool in box 310. As previously described, in some embodiments the formulation engine 80 processes information including, consumer 10 information, general product information, location information, and the like, to promote or reduce the importance of one or more beauty assessment parameters. In some embodiments, the formulation engine 80 receives the information from the cloud storage system 70. In some embodiments, the formulation engine 80 modifies the beauty assessment 22-1 or the step-by-step tutorial 22-2, or both, as previously described with respect to FIG. 1.

Many embodiments of the technology described above may take the form of computer- or controller-executable instructions, including routines executed by a programmable computer or controller. Those skilled in the relevant art will appreciate that the technology can be practiced on computer/controller systems other than those shown and described above. The technology can be embodied in a special-purpose computer, application specific integrated circuit (ASIC), controller or data processor that is specifically programmed, configured or constructed to perform one or more of the computer-executable instructions described above. Of course, any logic or algorithm described herein can be implemented in software or hardware, or a combination of software and hardware.

From the foregoing, it will be appreciated that specific embodiments of the technology have been described herein for purposes of illustration, but that various modifications may be made without deviating from the disclosure. Moreover, while various advantages and features associated with certain embodiments have been described above in the context of those embodiments, other embodiments may also exhibit such advantages and/or features, and not all embodiments need necessarily exhibit such advantages and/or features to fall within the scope of the technology. Accordingly, the disclosure can encompass other embodiments not expressly shown or described herein.

I/We claim:

1. A system for preparing a personalized cosmetic formula, the system comprising:
one or more beauty assessment devices;
a dispenser, comprising a plurality of mono-dose containers and a cosmetic formula packaging container, wherein individual mono-dose containers hold at least one ingredient;
a mixer that is compatible with the cosmetic formula packaging bottle shape; and
a digital hub configured to receive data from the one or more beauty assessment devices, send dispensing instructions to the dispenser, and send mixing instructions to the mixer.

2. The system of claim 1, further comprising a formulation engine 80 configured to modify a number of degrees of freedom measured by the one or more beauty assessment devices and to determine a routine of personalized cosmetic formulas.

3. The system of claim 1, wherein the one or more beauty assessment devices further comprise one or more sensors for at least one of: detecting, measuring, and receiving beauty assessment information.

4. The system of claim 1, further comprising a store screen, configured to receive personalized cosmetic formula information from the digital hub and to display the information.

5. The system of claim 1, wherein the cosmetic formula packaging bottle is pre-filled with at least one ingredient.

6. The system of claim 1, wherein the dispenser further comprises a control module, configured to implement a customized cosmetic formula under control from the digital hub.

7. The system of claim 1, wherein the mixer comprises an interlock.

8. The system of claim 7, wherein the mixer comprises a weighing stage.

9. The system of claim 1, wherein the mixer comprises one or more sensors, in communication with the digital hub.

10. The system of claim 1, wherein the mixer is configured to implement a mixing profile paired to the personalized cosmetic formula being prepared.

11. The system of claim 1, further comprising a printer engraving machine, configured to receive label information from the digital hub for creating a label for the personalized cosmetic formula packaging bottle.

12. The system of claim 1, further comprising a cloud storage system, configured to perform at least one of: receiving personalized cosmetic formula information from the hub, storing the personalized cosmetic formula information, and communicating the information to an external recipient.

13. A method for preparing a personalized cosmetic formula, comprising:
conducting beauty assessment;
communicating one or more results of the beauty assessment to a mobile digital hub;
creating a personalized cosmetic formula;
preparing a personalized cosmetic product by dispensing one or more mono-doses of active ingredients into a cosmetic formula packaging bottle, according to the personalized cosmetic formula;
mixing the personalized cosmetic product; and
labelling the personalized cosmetic product.

14. The method of claim 13, wherein creating a personalized cosmetic formula further comprises processing the results of multiple beauty assessments by a formulation engine to produce a routine that includes a series of personalized cosmetic formulae designed to achieve a desired cosmetic result over time.

15. The method of claim 13, further comprising attributing a unique identifier code to the customized formula.

16. The method of claim 13, further comprising communicating the personalized cosmetic formula to a dispenser, a mixer, and a compact printer engraving machine.

17. The method of claim 13, further comprising:
recording a measured weight the cosmetic formula packaging bottle before mixing;
comparing the measured weight against a calculated formula weight for the cosmetic formula packaging bottle; and
interlocking the mixer when the measured weight and the calculated formula weight are substantially unequal.

18. A non-transitory computer-readable medium having computer-executable instructions stored thereon that, in response to execution by one or more processors of a computing device, cause the computing device to perform actions comprising:
executing a beauty assessment;
processing a set of instructions and data produced by the beauty assessment;
controlling a dispenser; and
controlling a mixer.

19. The non-transitory computer-readable medium of claim 18, wherein executing the beauty assessment further comprises:
controlling a plurality of sensors; and
assessing cosmetic and personal information;

20. The non-transitory computer-readable medium of claim 18, wherein processing the set of instructions and data produced by the beauty assessment further comprises:
receiving cosmetic and personal information;
determining a formula;
assigning a formula identification code to the formula; and
transmitting the formula;

21. The non-transitory computer-readable medium of claim 20, wherein determining a formula further comprises:
receiving formulation data from at least one of a cloud storage system and a formulation engine.

22. The non-transitory computer-readable medium of claim 18, wherein controlling a dispenser further comprises:
effecting the dispensing of the contents of one or more mono-dose containers, individually holding one or more active ingredients, into a cosmetic formula container;

23. The non-transitory computer-readable medium of claim 18, wherein controlling a mixer further comprises:
receiving a measured weight of the contents of a cosmetic formula container and the container itself, combined;
comparing the measured weight against a calculated combined weight of the contents of the cosmetic formula container and the container itself;
controlling a mixer interlock;
determining a mixing profile;
applying the mixing profile; and
effecting the mixing of the contents of the cosmetic formula container according to the mixing profile;

24. The non-transitory computer-readable medium of claim 18, wherein the actions performed further comprise:
controlling a compact printer engraving machine to prepare a label for a cosmetic formula container.

25. The non-transitory computer-readable medium of claim 18, wherein the actions performed further comprise:
in communication with a cloud storage system, transmitting a formula identification code and a consumer profile.
